(19) United States
(12) Patent Application Publication
(10) Pub. No.: US 2026/0345266 A1
(43) Pub. Date: Nov. 5, 2026

(54) Autonomous Cobotic System for Enhanced Surgical Team Collaboration

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/432,305

(22) Filed: Dec. 24, 2025

Related U.S. Application Data
(63) Continuation-in-part of application No. 19/195,731, filed on Apr. 30, 2025.

(51) Int. Cl. A61B 34/32; B25J 9/16; B25J 11/00

(52) U.S. Cl. CPC A61B 34/32; B25J 9/1674; B25J 9/1679; B25J 11/0005

(57) ABSTRACT

A perioperative cobotic system is disclosed for enhancing collaboration among surgical team members. The system includes a mobile or semi-fixed cobotic platform with articulated arms, modular tool interfaces, and environmental sensors. A real-time team activity monitoring system uses visual, audio, and spatial sensing to identify team members and associated roles. A role-aware task allocation engine dynamically assigns and executes support tasks based on workload and procedural context. A multimodal communication interface accepts verbal, gesture, and touchscreen inputs and provides audio-visual feedback. A contextual awareness module identifies surgical phases and adapts cobotic behavior accordingly. Shared information displays present procedural data and coordination cues to local displays and augmented reality devices. Safety mechanisms enforce sterile-field compliance and proximity constraints. A learning engine enables team-specific adaptation, and a coordination module synchronizes personnel and tool readiness. Logged interactions support postoperative review and performance analysis.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to cobotic systems, and more specifically to a cobotic system that enhances surgical team coordination.

### BACKGROUND

Modern surgical procedures are conducted by multidisciplinary teams operating within high-stakes, dynamic, and spatially constrained environments. The effectiveness of a surgical intervention often depends not only on the skill of the lead surgeon, but also on the coordination and responsiveness of support personnel, including anesthesiologists, scrub nurses, circulating nurses, and surgical technologists. Despite technological advancements in instrumentation, imaging, and surgical navigation, intraoperative collaboration continues to rely heavily on verbal cues, manual handoffs, and ad hoc coordination strategies, which are vulnerable to communication lapses, misaligned timing, and inconsistent workflow execution.

Cobotic systems, robots designed to operate safely alongside human collaborators, have emerged as promising tools for assisting with surgical logistics, instrument handling, and support tasks. However, existing perioperative cobots primarily focus on isolated functionalities, such as tool delivery or autonomous mobility, and lack the integrated sensing, interpretive, and communication capabilities required for seamless collaboration with diverse surgical personnel. Current solutions do not adequately recognize the roles of individual team members, nor do they adapt behavior based on contextual understanding of team workload, procedure phase, or task urgency.

Moreover, operating rooms remain susceptible to delays caused by asynchronous arrival of personnel or tools, inefficient handoffs, and ambiguous task allocation. Conventional systems fail to synchronize surgical readiness at the start of a case and do not capture collaborative interactions for postoperative review or optimization. The absence of real-time coordination frameworks and shared situational awareness impairs workflow efficiency and limits the potential of robotic systems to proactively support human teams.

Accordingly, there is a need for a perioperative cobotic system that enables enhanced surgical team collaboration by combining role-aware task allocation, real-time team activity monitoring, multimodal communication interfaces, contextual awareness, and adaptive learning. Such a system should further include mechanisms for ensuring coordinated arrival of personnel and tools, enforcing sterile zone safety, and capturing procedural metrics to support auditability and continuous improvement.

### SUMMARY

The present disclosure relates to a perioperative cobotic system configured to enhance collaboration among members of a surgical team by integrating real-time sensing, adaptive behavior, and multimodal communication. The system comprises a collaborative cobotic platform equipped with one or more articulated robotic arms, modular tool interfaces, and a mobile or semi-fixed base that enables operation within the perioperative environment alongside human personnel.

A real-time team activity monitoring system includes visual, audio, and spatial sensors configured to detect, localize, and track surgical team members, differentiate among roles (e.g., surgeon, anesthesiologist, nurse), and inform task planning based on user proximity and functional context. A role-aware task allocation engine assigns and executes support tasks, such as instrument passing, lighting adjustments, or tray handling, based on predefined team responsibilities and real-time workload conditions.

The system further incorporates a multimodal communication interface that accepts verbal commands, gesture inputs, and touchscreen selections from team members, and provides audio-visual feedback confirming receipt and execution of tasks. A contextual awareness module identifies the current surgical phase, such as preparation, incision, or closure, and aligns cobotic actions to complement workflow progression without introducing interruption or redundancy.

Shared situational awareness is enabled by a display subsystem that projects relevant procedural information, team assignments, instrument readiness indicators, and alerts onto local screens or augmented reality headsets. Safety and compliance features include collision avoidance, sterile field recognition, and proximity alert systems configured to ensure safe operation and adherence to hospital protocols.

The system also includes a learning and adaptation engine configured to capture team-specific workflows and preferences over time, allowing the cobot to refine its task sequencing and communication style through repeated use. An audit trail and performance metrics module records interactions and outcomes to support postoperative review and quality assurance. Additionally, a coordination module ensures the timely arrival of essential personnel and tools prior to the scheduled start of surgery, promoting efficient case readiness.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of a perioperative cobotic system operating within an operating room environment.

FIG. 2 is a schematic block diagram of a real-time team activity monitoring system of the cobotic system.

FIG. 3 is a block diagram of a role-aware task allocation engine.

FIG. 4 is a diagram of a multimodal communication interface of the cobotic system.

FIG. 5A is a block diagram illustrating the contextual awareness module.

FIG. 5B is a flowchart illustrating operation of the contextual awareness module.

FIG. 6 is a diagram of a shared information display and coordination subsystem.

FIG. 7 is a diagram of an integrated safety and compliance subsystem.

FIG. 8 is a diagram of a learning and adaptation engine.

FIG. 9 is a schematic block diagram of a coordination module.

FIG. 10 is a diagram of an audit trail and performance metrics subsystem.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

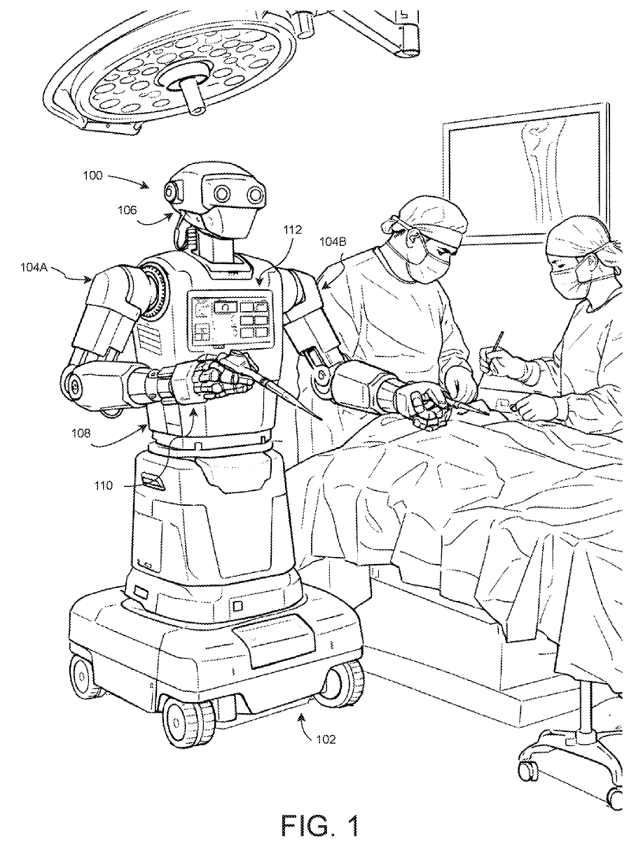

The following description, with reference to FIGS. 1-10, details an exemplary embodiment of a perioperative collaborative robotic system for enhanced surgical team collaboration. This integrated system is implemented as a humanoid cobot that operates throughout the perioperative environment (before, during, and after surgery) to assist the surgical team. For clarity, a single comprehensive embodiment is described, but it will be understood that various modifications and alternative configurations can be employed without departing from the scope of the invention. In the drawings, like parts may be indicated by like reference numerals for consistency across figures.

FIG. 1 illustrates a perspective view of the collaborative perioperative cobotic system 100 operating in an operating room environment alongside human surgical staff. The system 100 takes a humanoid form factor and is designed to work in a sterile or near-sterile environment. The cobot 100 includes a mobile base 102 supporting an upper body or torso 108, from which two articulated robotic arms 104A and 104B extend from shoulder-like joints on either side. A head unit 106 is mounted atop the torso and houses various sensors and communication interfaces (described further below). The mobile base 102 may be equipped with wheels or omni-directional rollers that enable the cobot to maneuver around the operating room floor with stability and precision. The base 102 contains drive motors, power supply (e.g., batteries or a tethered power cable), and a central computing module that runs the cobot’s control software. In one embodiment, the base 102 further includes a telescoping column or lift mechanism to adjust the height of the torso 108, allowing the cobot to reach high surfaces or equipment (e.g., overhead lights or supply shelves) and to approximate the height of human team members for better interaction.

The articulated arms 104A, 104B of system 100 provide multiple degrees of freedom (e.g., shoulder, elbow, wrist joints) enabling human-like reach and dexterity. Each arm terminates in an end-effector or hand 110 that is modular and interchangeable. For instance, the end-effector 110 can be a multi-fingered robotic hand capable of grasping surgical instruments, or a specialized tool attachment such as a gripper, clamp, suction device, drill or instrument tray holder depending on the task requirements. The cobot’s dexterous hands are designed to manipulate standard surgical tools and objects made for human use, which reduces the need for custom instruments, this is facilitated by fingered end-effectors with force and tactile sensors, coupled with optical cameras, to securely yet gently handle delicate items. The arms 104A, 104B and end-effectors 110 are configured with safety compliance (e.g., force-torque sensors at the joints) so that the robot can operate side-by-side with surgeons and staff without exerting dangerous forces even in the event of inadvertent contact.

In the embodiment of FIG. 1, the cobot 100 also carries an integrated user interface and communication panel 112 on its torso, above it’s shoulder, or as part of head unit 106. This interface 112 may include a touchscreen display and status indicator lights, enabling team members to receive visual information or quickly input commands (for example, a nurse could tap a request on the screen for the cobot to fetch a certain item). Speakers and microphones (not explicitly shown in FIG. 1) are also integrated, supporting verbal communication. The exterior of the cobot 100 is constructed of sterilizable or easily disinfected materials (e.g., a smooth polymer skin or removable sterile drapes over the robot’s surfaces) to allow operation in sterile environments without contamination. Additionally, the robot’s joints and actuators are sealed or shielded to prevent any particulate emissions or oil leaks into the sterile field. The humanoid shape of system 100 is advantageous in the surgical setting: it allows the cobot to navigate and interact with the environment similarly to a human, including pushing standard carts, opening doors, and positioning equipment, making its presence more intuitive and acceptable in the operating theater.

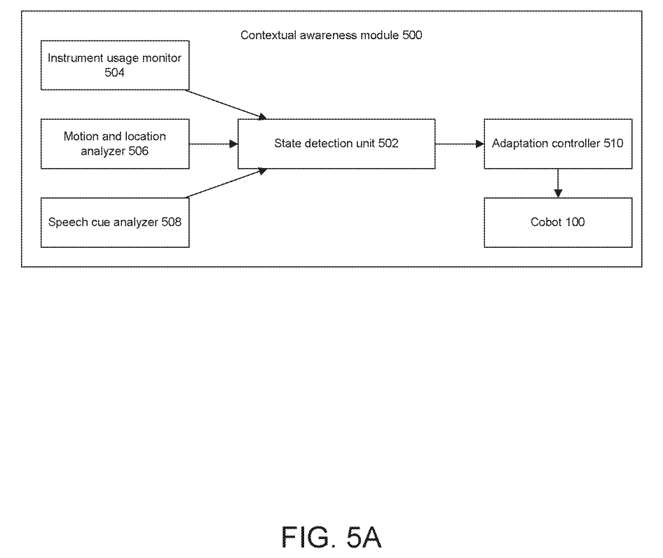

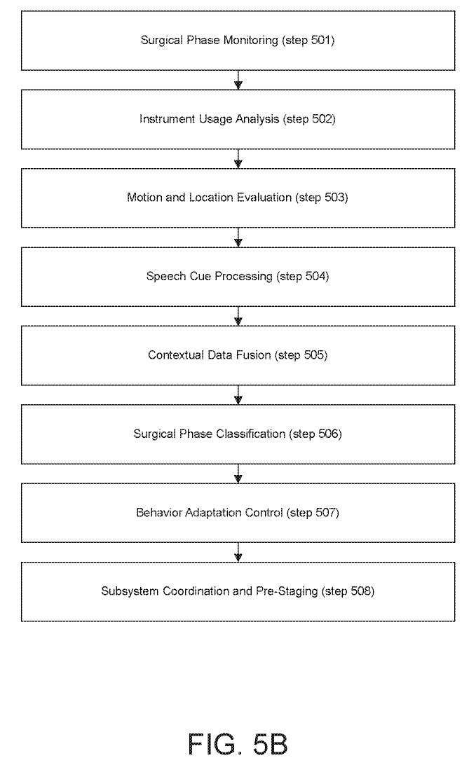

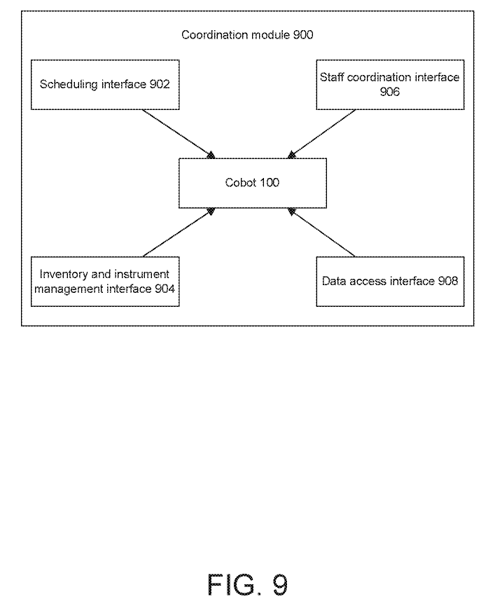

In typical use, prior to a surgical procedure, the cobot 100 positions itself in the operating room at a designated location (e.g., near the instrument table or at the periphery of the sterile field) and conducts initial system checks or self-calibration. Through coordination with hospital scheduling and inventory systems (see FIG. 9), the cobot ensures that all necessary personnel and tools are present and ready, helping to synchronize the team for a timely surgical start. During surgery, the cobot 100 remains alert and responsive: it works collaboratively by handing instruments, adjusting overhead lighting or other equipment, and retrieving additional supplies as needed, all in concert with the human team’s workflow. It maintains a safe distance from the sterile field unless required to momentarily reach in with a sterile tool, and its movement and actions are continuously modulated based on the surgical phase and real-time feedback (see FIG. 5). After the procedure (e.g., during closing and post-operative phases), the cobot can assist with cleanup and turnover tasks, such as holding a disposal bin for used instruments, helping count instruments and sponges for audit, or preparing the room for the next surgery. Throughout all these phases, the various subsystems of the cobot function together seamlessly as described in further detail below.

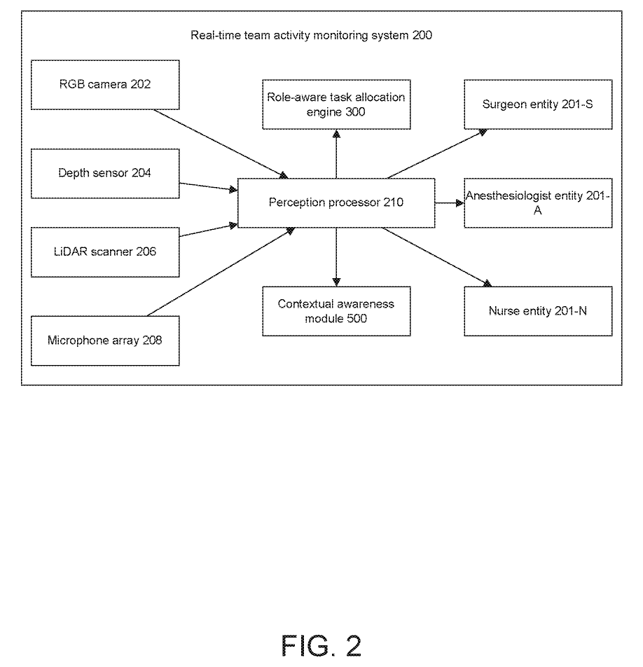

FIG. 2 shows a schematic diagram of the real-time team activity monitoring system 200 of the cobot. This subsystem enables the cobot 100 to perceive and understand the positions and actions of the surgical team members in the operating room. In the illustrated embodiment, the monitoring system 200 comprises an array of visual, audio, and spatial sensors. One or more high-resolution RGB cameras 202 are mounted on the cobot’s head unit 106 (and/or on the torso 108) to capture images and video of the environment. These cameras 202 may be arranged to provide a wide field of view or even a full 360° coverage when combined, ensuring that all key areas of the OR (operating room) are within view. Depth sensing cameras or sensors 204 (such as stereo cameras or time-of-flight depth sensors) are also included to perceive three-dimensional positions of people and objects. In addition, a LiDAR scanner 206 or similar scanning laser rangefinder can be integrated on the cobot (for example, near the top of the head or on the base) to map the room in 3D and track movement with high spatial precision even in low-light or occluded conditions.

The monitoring system 200 further includes an array of microphones 208, for example placed around the head unit 106 or on the torso, to capture audio signals from multiple directions. The audio data is processed to perform speech recognition and detect auditory cues (such as a surgeon’s verbal request or an alarm sound from medical equipment). In some embodiments, the cobot may also incorporate wearable sensor inputs, such as receiving signals from RFID tags or ultra-wideband beacons worn by staff, to augment the vision-based identification of personnel. All these sensor inputs (visual, depth, LiDAR, audio, etc.) are fused in real-time by a perception processor 210 (a computing unit running on the cobot’s central computer in the base 102 or a distributed set of processors). The perception processor 210 runs advanced algorithms, including computer vision models (potentially employing machine learning), to detect and identify each person in the room and to recognize their role and activities. For instance, using the video feeds, the system can distinguish the lead surgeon, assistant surgeon, scrub nurse, circulating nurse, anesthesiologist, and so on, based on their attire, location, and learned facial or body recognition if permissible. Each identified person is tracked as an entity with a role label and spatial coordinates within the OR map that the cobot maintains.

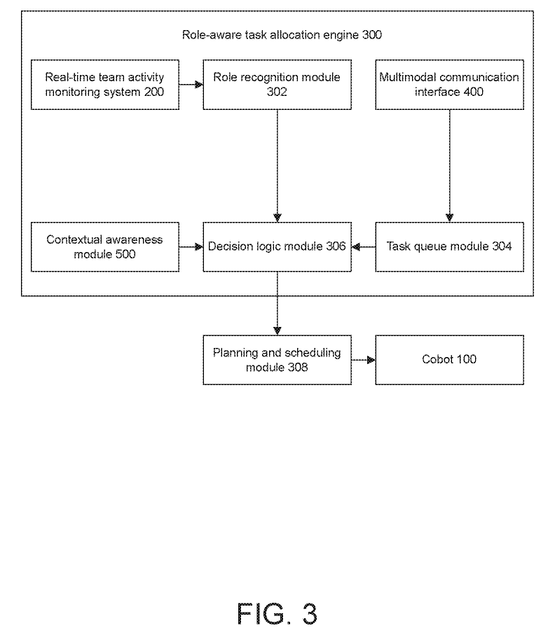

Through analysis of gestures and posture, the monitoring system 200 can interpret team members’ behavior. For example, if the surgeon (identified as entity 201-S) extends an open hand toward the side, the vision system recognizes this as the surgeon expecting an instrument. If the anesthesiologist (entity 201-A) turns toward the anesthesia cart, the system notes that their attention is on patient vitals or medication. If a circulating nurse (entity 201-N) moves toward a supply cabinet, the cobot perceives a supply retrieval action. The monitoring system 200 updates each person’s state continuously, such as “surgeon is operating at the surgical site,” “scrub nurse is preparing the next instrument,” or “anesthesiologist is adjusting oxygen levels.” This rich situational awareness data is then provided to other subsystems, notably the role-aware task allocation engine (FIG. 3) and the contextual phase detection module (FIG. 5). By having a constant, multi-modal understanding of the team’s activities and needs, the cobot creates a foundation for truly proactive and context-sensitive assistance.

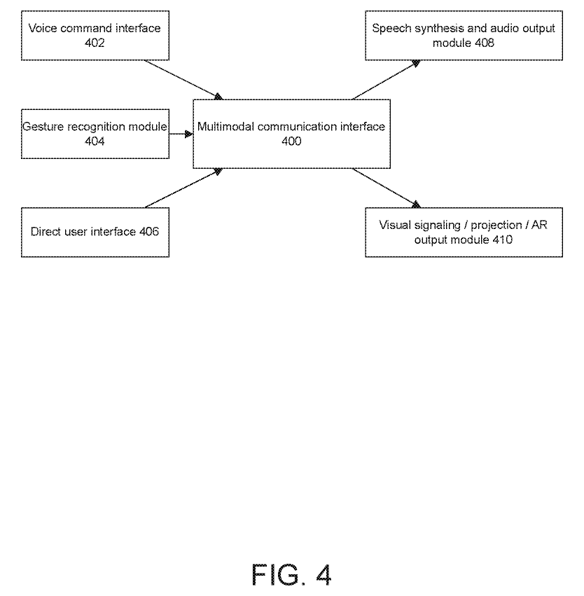

FIG. 3 is a block diagram illustrating the role-aware task allocation engine 300, which orchestrates how tasks are assigned among the cobot 100 and the human team members dynamically. The engine 300 resides in the cobot’s control system (e.g., within the central computing module in base 102) and continuously receives inputs from the team monitoring system 200 (FIG. 2) as well as other context inputs such as the surgical phase (from FIG. 5) and any direct commands (from the interfaces described with FIG. 4). In one embodiment, the task allocation engine 300 comprises several functional modules. A role recognition module 302 maintains data on the roles and current status of each team member (as identified by the monitoring system). A task queue module 304 stores pending tasks or requests that need addressing – tasks might be generated autonomously by the cobot (for example, detecting that an instrument is needed or that an overhead light is out of position) or come from user input (such as a verbal request from the surgeon or a tap on the interface 112).

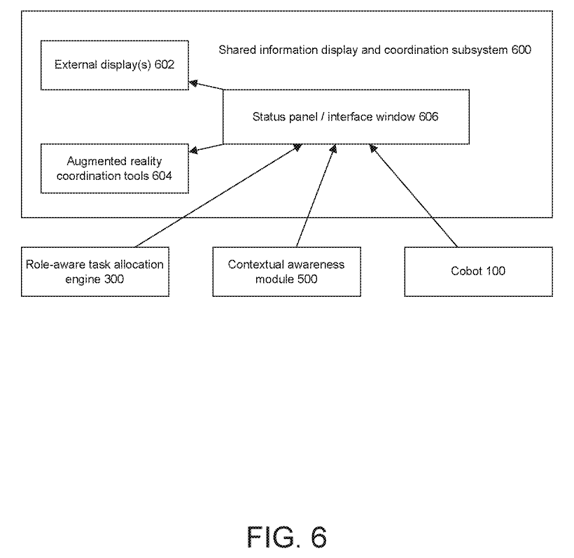

The engine 300 further includes a decision logic module 306 that analyzes the active situation and decides who (or what subsystem) should carry out each task. The decision logic 306 may use a set of predefined rules and priorities, and in some cases machine learning models or an expert system, to evaluate factors such as: the criticality and urgency of the task, the current workload on each human team member, the role competencies (for instance, only the anesthesiologist should administer medications, the cobot or a nurse can fetch equipment, etc.), and the physical proximity of the cobot or personnel to the task location. Based on this evaluation, the engine 300 assigns tasks either to a human (by notifying them via the shared interface of FIG. 6) or to the cobot’s own execution queue. For example, if the surgeon’s hand is outstretched requesting an instrument, the engine recognizes this and might immediately task the cobot to pick up the requested instrument from the instrument tray and place it in the surgeon’s hand (if the scrub nurse is occupied or if the system is explicitly set to handle instrument passing). As another example, if an overhead surgical lamp needs re-positioning, the engine 300 can decide to send that task to the cobot, which can either adjust a connected smart lamp via a network command or physically reach up and adjust it using an arm 104 if within reach. Similarly, when routine supply retrieval is needed (e.g., a new suture pack), the engine might dispatch the cobot to retrieve it from a nearby table or pass the instruction to a circulating nurse depending on who is less busy.

Task allocation engine 300 works in real-time and updates assignments continuously. A planning and scheduling module 308 interfaces with the cobot’s motion control to execute the tasks the cobot is assigned, managing any sequence of actions required. For instance, to pass an instrument, module 308 will coordinate the arm motion, grasping the instrument with the appropriate end-effector 110, and use the sensor feedback to hand it over smoothly when the surgeon grasps it. Importantly, the engine 300 is role-aware: it understands not only what needs to be done, but also which roles are responsible or best suited for each task. If an unexpected situation arises (for example, an emergency where the surgeon needs to focus completely on the patient), the engine can escalate more tasks to the cobot (and other staff) to relieve the surgeon of anything non-critical to the operation. Conversely, if the cobot is at its capacity, the engine knows to not overload it and instead will prompt human team members. This dynamic task-sharing optimizes team efficiency and helps ensure that every task, whether it is passing an instrument, adjusting an apparatus, or fetching supplies, is handled by the most appropriate agent available.

FIG. 4 illustrates the multimodal communication interface 400 that enables intuitive interaction between the surgical team and the cobot 100. Because operating rooms are busy and sometimes noisy environments, the system 100 provides multiple parallel channels for communication to ensure commands and information can be exchanged effectively. One component is a voice command interface 402, which utilizes the microphones 208 and an onboard speech recognition unit. In operation, team members can address the cobot with simple verbal commands or queries (for example, “cobot, increase table light,” or “bring the suture kit”). The speech recognition module 402 parses these commands in real-time and the cobot’s control system confirms and executes them when recognized. The voice interface is designed with a specialized vocabulary and language model tuned to medical contexts and the specific terminology of surgical procedures, to improve accuracy even with technical terms or abbreviations. In some embodiments, the cobot may also distinguish different speakers or require a wake word (“Hey Cobot”) to avoid confusion with background conversation.

In addition to voice, the interface 400 includes a gesture recognition module 404. The cameras 202 and depth sensors 204 feed this module, allowing the cobot to interpret hand gestures or body language cues from the team. For instance, a simple hand wave might get the cobot’s attention, an open hand could mean a request for an item, or a thumbs-up might signal confirmation. The gesture library can be customized to the preferences of the surgical team or to standard gestures defined by hospital protocol. The system uses computer vision algorithms to track human hand positions and poses, and can function in tandem with voice commands (e.g., a surgeon could combine a verbal “give me the retractor” with a hand gesture pointing to the retractor, providing redundant cues for clarity).

The communication interface 400 further encompasses direct user interface elements 406 such as the touchscreen or control panel 112 on the cobot, and potentially a remote tablet or workstation interface accessible to support staff. Through this interface 406, users can manually input requests or parameters, such as selecting from a menu of common tasks (“Prepare suction device”, “Fetch more gauze”) or adjusting settings (like the volume of the cobot’s voice feedback or the sensitivity of certain alerts). Visual output from the cobot is also provided: the display on interface 406 can show contextual prompts (e.g., “Ready to receive instrument” or “Task in progress: sterilizing tool”), or it can be used to show live video feeds if the cobot needs to share what it is seeing (for example, highlighting a dropped instrument under a table).

Completing the multimodal interface 400 are the cobot’s output communication means: a speech synthesis and audio output module 408 (with speakers behind a grille on head 106 or torso 108), and an integrated lighting or projection system 410 for visual signals. The speech synthesis 408 allows the cobot to speak to the team in a polite, clear manner, for instance, announcing “Instrument sterilized and ready” or giving a brief alert “Warning: obstacle detected behind you.” It can modulate tone and volume depending on urgency (e.g., quiet confirmation vs. louder alert). The visual signaling 410 may include status lights (for example, a green light indicating idle/ready status, yellow for busy, red for an urgent condition), or even a small projector that can cast simple symbols or directions onto surfaces (for example, projecting an arrow on the floor to show it is headed to the supply cabinet). In some embodiments, module 410 also represents an augmented reality (AR) interface that works with AR glasses worn by staff: the cobot’s system can send real-time overlays or notifications to those glasses (see also FIG. 6 regarding shared display), such as highlighting the exact location where a tool is to be placed. By providing voice, gesture, touch, and visual communication modalities together, the interface 400 ensures robust and flexible interaction, accommodating individual preferences and situational demands. If one mode is impaired (for example, the room is too noisy for voice or the surgeon’s hands are occupied so they cannot use gestures), another mode can fill in, thereby greatly enhancing the human-cobot collaboration.

FIG. 5A is a block diagram illustrating the contextual awareness module 500 of the perioperative cobotic system. The contextual awareness module 500 is configured to detect, classify, and respond to distinct operative phases of a surgical procedure in order to adapt behavior of the cobot 100 based on real-time procedural context and anticipated workflow needs. The contextual awareness module 500 includes a state detection unit 502, an instrument usage monitor 504, a motion and location analyzer 506, a speech cue analyzer 508, and an adaptation controller 510. These components operate cooperatively to analyze multimodal contextual inputs associated with surgical team activity, tool usage, spatial movement, and verbal cues, and to generate phase-aware control outputs for the cobot 100. Each operative phase, including pre-operative preparation, anesthesia induction, incision start, active intervention, closing, and post-operative wrap-up, is associated with characteristic activity patterns and workflow priorities that are represented within the contextual awareness module 500.

The state detection unit 502 functions as a central aggregation and evaluation component within the contextual awareness module 500 and receives inputs from the instrument usage monitor 504, the motion and location analyzer 506, and the speech cue analyzer 508. The instrument usage monitor 504 is configured to track surgical instruments in use and evaluate instrument identity, frequency of use, sequencing, and dwell times. The motion and location analyzer 506 is configured to evaluate spatial positions, movements, and postures of surgical team members and, where applicable, a patient. The speech cue analyzer 508 is configured to process audio input to detect predefined verbal cues or structured verbal events indicative of surgical phase transitions. Outputs of these components are provided to the state detection unit 502, which generates a contextual state representation that is supplied to the adaptation controller 510.

The adaptation controller 510 is configured to adjust cobot operating parameters and available actions based on the determined surgical phase. Such adjustments may include modifying motion speed, suppressing non-urgent alerts, altering communication modes, enabling or disabling certain task behaviors, and coordinating with other system modules to prepare for upcoming workflow stages. Through this structural arrangement, the contextual awareness module 500 provides phase-aware control of the cobot 100.

FIG. 5B is a flowchart illustrating operation of the contextual awareness module 500 in accordance with the block diagram of FIG. 5A. The flowchart represents an example method by which surgical phase detection and behavior adaptation are performed during a surgical procedure.

Surgical Phase Monitoring (step 501). In step (501), contextual inputs associated with a surgical procedure are monitored to support determination of a current operative phase. The monitoring is performed continuously and in parallel with other cobot subsystems.

Instrument Usage Analysis (step 502). In step (502), surgical instrument usage data is analyzed, including instrument identity, frequency of use, sequencing, and dwell times. Instrument usage patterns are evaluated for correlation with particular surgical phases.

Motion and Location Evaluation (step 503). In step (503), spatial positions, movements, and postures of surgical team members and, where applicable, a patient are evaluated. Detected motion patterns and positional changes are analyzed for indications of phase transitions or workflow inflection points.

Speech Cue Processing (step 504). In step (504), audio input is processed to detect predefined verbal cues or structured verbal events associated with surgical phase transitions. Detected cues are correlated with other contextual inputs.

Contextual Data Fusion (step 505). In step (505), data derived from instrument usage analysis, motion and location evaluation, and speech cue processing is fused to produce a unified contextual state representation.

Surgical Phase Classification (step 506). In step (506), a current surgical phase is classified based on the unified contextual state representation. Classification may be performed using a rule-based state machine or a trained machine learning model.

Behavior Adaptation Control (step 507). In step (507), operating parameters and available actions of the cobot 100 are adjusted in accordance with the classified surgical phase, including modifying motion behavior, communication modes, and task execution priorities.

Subsystem Coordination and Pre-Staging (step 508). In step (508), coordination actions are initiated based on predicted workflow progression, including pre-staging tasks, preparing for subsequent procedural phases, or initiating readiness activities for a next surgical procedure.

Through execution of steps (501)–(508), the contextual awareness module 500 enables the cobot 100 to operate in a phase-appropriate and anticipatory manner, reducing workflow disruption and supporting efficient collaboration with the surgical team.

FIG. 6 illustrates the shared information display and coordination subsystem 600 provided by the cobot to keep the surgical team informed and synchronized. In one embodiment, the system 600 includes one or more external displays 602 in the operating room that the cobot can control or send data to. These could be existing OR wall monitors or a holographic projector system that the hospital uses for surgical data. The cobot links to these displays 602 wirelessly or via cable and can present real-time information such as task statuses, upcoming steps, or checklists. For example, a large monitor might show a dynamic checklist of surgical milestones or requirements (like “Time Out completed, Antibiotics given, All tools sterilized, Imaging ready” with green checkmarks), which the cobot updates as those tasks are done. It could also display which team member or the cobot is responsible for the next step (e.g., “Next: Administer heparin – Anesthesiologist” or “Next: Place instrument tray – Cobot”). This ensures the whole team shares a mental model of the workflow.

Additionally, the subsystem 600 features augmented reality (AR) coordination tools 604. If the surgical staff are equipped with AR headsets or smart glasses, the cobot can send tailored visual overlays to each person. For instance, through an AR headset worn by the circulating nurse, the cobot might highlight the location of a specific supply needed and display a pointer or path to it (making it faster for the nurse to fetch). A surgeon’s AR view could get a subtle notification that the cobot is about to adjust the lighting or that a requested instrument is en route to their hand, without the need for verbal confirmation. In embodiments without AR glasses, an overhead projector or a pointer laser could be used for simpler cues (like spotlighting an instrument on the table that has been requested).

The coordination tools also include a status panel or interface window 606 that can be accessed on the cobot’s own display 112 or a connected tablet. This panel provides a more text-based or tabular view of system status and assignments, for example, listing all active tasks, who/what is handling them, and any pending requests. Team members (especially the circulating nurse or OR coordinator) can glance at this to verify that nothing is overlooked. The cobot updates this information in real time as directed by the task allocation engine (FIG. 3) and as phases change (FIG. 5). Furthermore, the cobot can use the display 602 or AR 604 to convey safety or timing information: for example, a countdown timer before a critical phase (like “5 minutes to cardiopulmonary bypass ready”), or an alert if a preparation task is lagging (highlighting it in red on the checklist). All shared information displays are designed to complement existing OR communication protocols, not replace them, so the system can be configured to only display certain types of information to avoid information overload. By giving every team member immediate insight into what the cobot is doing and what tasks are in progress or upcoming, subsystem 600 helps prevent miscommunications and keeps the surgical workflow running smoothly.

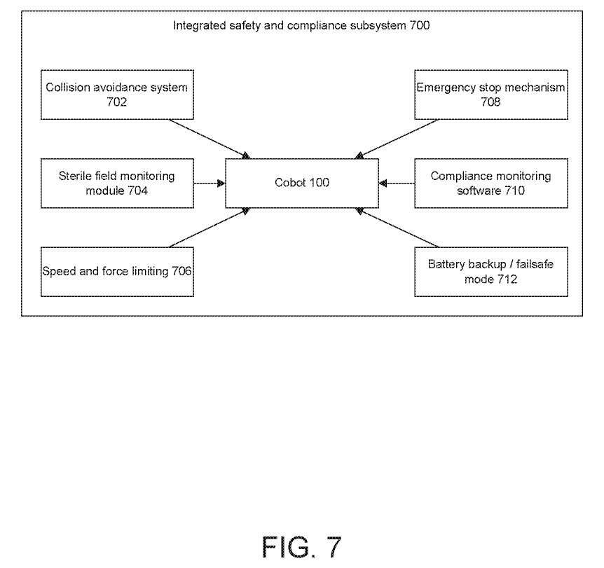

FIG. 7 depicts the integrated safety and compliance subsystem 700 of the cobot, which ensures that all robotic operations are conducted safely and in accordance with medical regulations and sterile practices. Because the cobot 100 operates in close proximity to patients and clinicians, safety is paramount and is enforced through multiple layers of hardware and software safeguards. One key component is a collision avoidance system 702. This system uses inputs from range sensors (such as the depth sensors 204 and LiDAR 206 from FIG. 2) to detect obstacles or people in the cobot’s path in real time. The motion planning software of the cobot constantly computes a safe trajectory for any movement of the base 102 or arms 104A, 104B, and if an unexpected obstacle enters the path (e.g., a nurse stepping over to the patient quickly), the collision avoidance 702 either stops or reroutes the movement immediately. The arms are programmed to yield if they encounter resistance beyond a calibrated threshold, this compliance, aided by force sensors, means if the arm brushes against a person or object, it will halt or back off rather than push through.

Another critical safety feature shown in FIG. 7 is a sterile field monitoring module 704. This module ensures the cobot respects the boundaries of the sterile field (the area around the patient and instrument table that must remain sterile). The module 704 can utilize visual processing to recognize the sterile drapes and the position of the patient, effectively mapping out a forbidden zone that the non-sterile parts of the robot (such as its base 102 and any non-sterile arm segments) must not cross. In one implementation, special markers or sensors (like infrared beacons or RFID tags) are placed at sterile boundary points (on drapes or floor) and corresponding receivers on the cobot detect their presence to delineate the sterile zone. The cobot’s control system then imposes virtual constraints: for instance, the base 102 will not drive into the sterile region, and arms 104 will only extend into that area if they have a sterile cover or are handing a sterile tool, and even then, with minimal intrusion. Module 704 works closely with collision avoidance 702 to treat the sterile field as an immovable obstacle that must not be violated.

The compliance features further include speed and force limiting 706. When the cobot operates near humans (which is essentially at all times in the OR), its joints and base limit their maximum speed and acceleration to safe levels. During delicate phases, the adaptation controller (FIG. 5) may command even lower speed settings. If the cobot needs to hand over a sharp instrument, it approaches very slowly and pauses if it does not sense a steady grasp from the recipient. Force limiting is achieved by monitoring motor currents and dedicated force/torque sensors; if any axis measures above a threshold force (such as if an arm pushes against something unexpectedly), the control loop triggers an immediate stop and retreat.

Additionally, an emergency stop mechanism 708 is provided as part of subsystem 700. This includes both physical emergency stop buttons on the cobot’s body that any team member can press to instantly cut power to the motors, and a voice-activated emergency phrase that the speech system can recognize (for example, if someone yells “Stop robot!”). The system can also be tied into the operating room’s master alarm system to automatically stop if certain conditions occur (like defibrillation events or other hazards where the robot must be stationary).

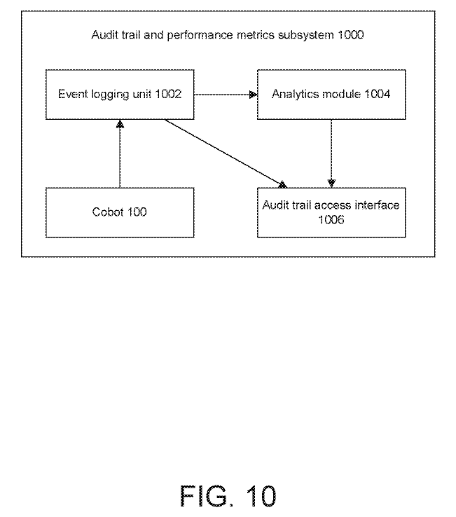

Compliance with hospital and regulatory standards is also built-in. The cobot’s software 710 keeps logs for safety compliance (shared with FIG. 10’s audit trail) and monitors itself for any faults (performing self-checks on sensors and actuators periodically). The materials used are biocompatible where needed and can withstand hospital-grade cleaning and sterilization processes. The design may include covers or shields so that any heat or electrical components cannot accidentally come into contact with the patient or staff. The cobot also has battery backup or failsafe modes 712: in the event of power loss or system fault, it can smoothly halt and hold position (so it doesn’t, for instance, drop an instrument). Through the combination of collision avoidance, sterile field adherence, speed/force limits, emergency stops, and compliance checks, the subsystem 700 ensures that the introduction of the cobot into the surgical environment does not introduce new risks, but rather operates as a trustworthy and safe assistant at all times.

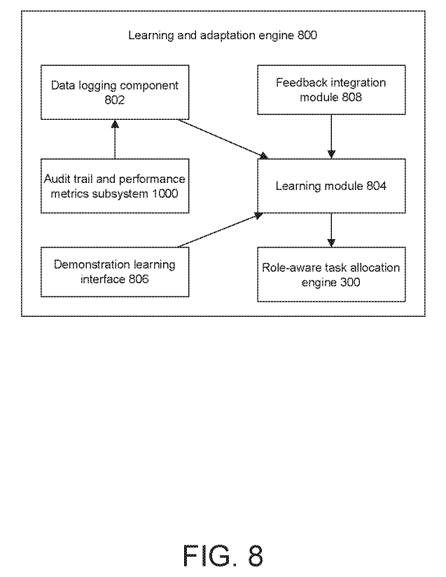

FIG. 8 is a diagram of the learning and adaptation engine 800 of the cobot system, which enables continuous improvement of its performance and the customization of its behavior to specific surgical teams or procedures. The engine 800 includes a data logging component 802 that gathers rich data during each operation. This data includes sequences of tasks performed, timing information, outcomes (e.g., whether a task was successful on first attempt or if a human had to intervene), and contextual information (such as which surgical team was involved, what procedure it was, and any unusual events). Key interactions, like every time the cobot passes an instrument or adjusts equipment, can be recorded with relevant parameters (time, any delay, any errors, etc.). Additionally, data on preferences (e.g., surgeon’s preferred instrument hand-off technique or anesthesiologist’s preferred timing for medication alerts) are noted.

All of this logged data is used by a learning module 804. The learning module 804 may incorporate machine learning algorithms, such as reinforcement learning or supervised learning from demonstration, to analyze performance and identify ways to improve. In one mode, the system can perform offline analysis after each surgery: for instance, it can analyze if there were any patterns where the cobot’s assistance was slower than ideal, or if there were moments of inactivity where it could have helped but did not. Using this analysis, the module 804 adjusts the underlying models or parameters of the task allocation engine (FIG. 3) and other subsystems. For example, if it learns that a particular surgeon often expects a certain instrument without verbally asking (perhaps they always reach for it at a predictable step), the cobot can learn to anticipate that need in future surgeries with that surgeon, readying the instrument proactively. Similarly, the module could fine-tune the gesture recognition (FIG. 4) by learning the specific nuances of how a person gestures (some surgeons might make a very subtle hand motion which the system then learns to recognize as a valid request). In an embodiment this information is used to create a digital twin of the surgeon and/or the OR.

Another part of engine 800 is a demonstration learning interface 806. This allows human team members or trainers to explicitly teach the cobot new tasks or preferences. One approach is via kinesthetic teaching: a person can take hold of the cobot’s arm (with safety mode active) and physically guide it through a motion, such as how to properly position a particular retractor or how to perform a certain cleaning sweep on the floor. The sensors record this demonstration and the control software refines its movement planning based on that example. Another approach is through a training mode where the cobot observes a task being done by a human and then imitates it under supervision. The demonstration interface 806 might include a special “learning mode” UI on the touchscreen 112 to start or stop a demonstration recording, and options to label or annotate the taught behavior (e.g., “This is how to arrange instruments for surgeon Dr. X’s preference”).

The adaptation engine 800 also contains a feedback integration module 808. This module can take direct feedback from users, such as ratings or corrections (for instance, after a surgery, the surgeon or staff might provide feedback: “the cobot moved too close to the sterile field at one point” or “it reacted slowly to a command”). The module 808 uses this human feedback to adjust thresholds or behavior rules (perhaps making the cobot more conservative around the sterile field if noted, or increasing the alertness level). Over time, as the cobot participates in more procedures, it builds a refined model of how to best assist in various scenarios and for various personal styles. These improvements are stored in its knowledge base and can even be shared across multiple units or ORs in a hospital (subject to privacy and security policies) so that if one cobot learns a new best practice, others can update to match. The end result of the learning and adaptation engine 800 is a system that does not remain static, but rather evolves to become more efficient, more user-friendly, and more tightly integrated into the surgical team’s way of working as experience grows.

FIG. 9 is a schematic block diagram of the coordination module 900, through which the cobot interfaces with broader hospital information systems and resources. This module 900 allows the cobot to extend its collaborative reach beyond the immediate operating room by tapping into data and services that support the perioperative process. A scheduling interface 902 connects the cobot to the hospital’s OR scheduling system or electronic health record (EHR) scheduling component. Via this interface, the cobot retrieves the day’s surgical schedule, including details like procedure type, expected start and end times, assigned personnel, and patient information (within allowed privacy constraints). By knowing the schedule, the cobot can proactively prepare for upcoming cases, for example, if a case is delayed or moved to another OR, the cobot will receive that update and adjust its plan (such as moving to the correct room or delaying certain preparatory tasks).

The coordination module 900 also includes an inventory and instrument management interface 904. This interface ties into the hospital’s inventory databases or the sterile processing department’s tracking system (for instance, systems that use barcodes or RFID tags to track instrument sets and surgical supplies). Through interface 904, the cobot can verify that the required surgical instrument kit and any special equipment for the procedure are available and have completed sterilization. If an item is missing or not yet ready, the cobot can alert staff in advance rather than at the last minute. In some embodiments, the cobot can physically assist in fetching needed items from nearby storage if they are not present, by using its mobile base 102 and potentially communicating with automated pharmacy or supply robots via this same module. The coordination module can signal to hospital staff or other robots to deliver an item if it’s stored remotely.

A further component is the staff coordination interface 906, which can integrate with systems like staff paging or communication systems. This interface can be used before surgery to ensure all assigned team members are on site and ready. For instance, if the schedule says a particular specialist is needed but the monitoring system (FIG. 2) hasn’t detected their presence by a certain time, the cobot through module 900 could send a page or message via the hospital system to remind them or notify the OR coordinator. Similarly, if a surgery is ahead of schedule, the cobot can coordinate to have the next patient or team prepared sooner.

Module 900 may also include a data access interface 908 for pulling in relevant patient or procedure-specific data. For example, through a secure connection to the patient’s electronic health record or anesthesia record (with proper authorization), the cobot might retrieve data like known allergies (which could be relevant for handing certain materials), or the surgical plan notes (to know if any special implants will be used that might need preparation). This data can further refine the cobot’s assistance; for instance, if the plan indicates that two units of blood should be on standby due to anticipated blood loss, the cobot can double-check via inventory interface 904 that those are ready, or remind the team via the shared display if needed.

All communications in the coordination module 900 are secured and compliant with privacy regulations (like HIPAA in the US), meaning patient identifiers are handled carefully and only necessary information is used. The cobot essentially acts as another node on the hospital’s network that participates in routine workflows. By connecting scheduling, inventory, and staff communications into its cognitive loop, the cobot not only helps within the OR but also smooths the transitions and logistics around the surgery. For example, just before a case starts, it can confirm everything is set (tools, people, patient) and after a case, it might signal housekeeping to clean the room once it detects everyone has left. The coordination module thus ensures the cobot is an active participant in the larger perioperative ecosystem, bridging the gap between the immediate surgical procedure and the hospital’s support infrastructure.

FIG. 10 illustrates the audit trail and performance metrics subsystem 1000 of the cobot, which records data from operations and provides analytical insights into team collaboration and system performance. The subsystem 1000 includes an event logging unit 1002. During each procedure, this unit 1002 automatically documents a timeline of notable events and actions involving the cobot and potentially other team actions as observed. For example, entries in the log may include: “[10:15:20] Cobot fetched Tool X from supply cart; [10:15:30] Cobot passed Tool X to Surgeon; [10:45:00] Cobot issued safety alert: non-sterile approach detected; [11:00:00] Surgery complete, cobot began cleanup assistance.” Each log entry can be timestamped and labeled with relevant details (which person was involved, which tool, etc.). The log also integrates data like changes in surgical phase or task allocation decisions (e.g., “task Y reassigned to nurse due to cobot workload”).

In addition to raw event logging, the subsystem 1000 computes performance metrics via an analytics module 1004. This module processes the log data and possibly the sensor data to derive quantitative measures. Metrics might include the number of tasks completed by the cobot per surgery, the average response time to a surgeon’s request, the amount of idle time where the cobot was available but not utilized, the adherence to schedule (for instance, did the cobot help avoid delays by ensuring prompt starts), and safety metrics such as number of collision close-calls or boundary violations avoided. Team collaboration metrics can also be derived, like how often the cobot stepped in to help a certain role (maybe indicating that role was understaffed or overburdened), or the ratio of tasks handled by cobot vs humans. These metrics are valuable for process improvement: for example, if the cobot’s task coverage is low, it may indicate underutilization or trust issues to be addressed with training; if the response time is high, the system might need tuning or the staff might need more familiarity with how to cue the cobot faster.

The audit trail data can be accessed after the surgery via a secure interface 1006. This might be a web dashboard or part of the hospital’s quality assurance system. Surgeons or administrators can review the timeline of events for a particular case to reconstruct who did what and when – a tool that can be invaluable for post-operative debriefs or investigating any incidents. The timeline could be visualized alongside the human team’s actions to see the integration. This fosters accountability and transparency for the cobot’s actions; since every automated action is logged, there is always a record. Moreover, the performance metrics might be aggregated over multiple surgeries to track trends: for example, a monthly report could show that “use of the cobot has reduced average surgery prep time by 10 minutes” or that “no safety incidents occurred in X hours of operation”.

Finally, the audit subsystem 1000 feeds back into the learning engine (FIG. 8) by highlighting areas for improvement. If certain tasks consistently show longer completion times or certain alerts are frequent, those become targets for the adaptation algorithms to refine. In some embodiments, the system may also allow a comparison between different teams or configurations (anonymously) to identify best practices. For instance, one surgical team might achieve faster instrument hand-offs with the cobot, indicating their way of interacting could be taught to others. All these audit and metric capabilities ensure that the cobot’s integration into surgical workflow remains under continuous evaluation, meeting high standards of performance and safety, and that the benefits to collaboration can be quantified and improved over time.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A perioperative cobotic system for enhanced surgical team collaboration, comprising:
a cobot including a mobile or semi-fixed base, at least one articulated robotic arm, and one or more environmental sensors, the platform being configured to operate safely alongside human surgical team members in a perioperative environment;
a real-time team activity monitoring system comprising at least one visual sensor, at least one audio sensor, and at least one spatial sensor, the monitoring system configured to detect, localize, and track surgical team members, to differentiate among roles of the team members, and to adapt the system’s behavior based on the identified roles;
a role-aware task allocation engine configured to dynamically assign and execute support tasks based on predefined role responsibilities of the surgical team members and real-time workload assessments;
a multimodal communication interface configured to receive verbal commands, interpret hand gestures, and accept touchscreen inputs from the surgical team, and further configured to provide audio and visual feedback acknowledging commands and conveying status information;
a contextual awareness module configured to identify a current phase of a surgical procedure and align the cobotic system’s behavior with workflow timing corresponding to the identified surgical phase;
a shared information display subsystem configured to provide surgical data and team coordination cues to at least one local display and at least one augmented reality headset for viewing by the surgical team;
a safety and compliance control subsystem configured to implement collision avoidance, sterile-field recognition, and proximity alert protocols so that the cobot operates in compliance with operating room safety standards and does not interfere with sterile areas or personnel;
a learning and adaptation engine configured to capture team-specific workflow patterns and preference data and to improve the cobotic system’s performance over time based on accumulated experience;
an audit trail and performance metrics module configured to log interactions between the cobotic system and the surgical team and to generate postoperative collaboration reports summarizing team performance metrics; and
a coordination module configured to ensure that designated surgical personnel and required surgical tools are present in the operating room by a scheduled surgical start time to minimize idle delays.

2. The system of claim 1, wherein the real-time team activity monitoring system comprises an optical camera as the visual sensor, a microphone as the audio sensor, and a depth sensor as the spatial sensor, and wherein the monitoring system is further configured to identify individual team members and associate each identified team member with a role selected from a group consisting of surgeon, anesthesiologist, scrub nurse, and circulating nurse.

3. The system of claim 1, wherein the role-aware task allocation engine assigns support tasks including passing a surgical instrument to a surgeon, adjusting an operating room light, and managing a surgical instrument tray, the engine being configured to dynamically reprioritize or reassign tasks in response to changes in team workload or spoken requests from the surgical team.

4. The system of claim 1, wherein the multimodal communication interface comprises:
a voice recognition module configured to interpret verbal commands issued by surgical team members;
a gesture sensing module configured to detect predefined hand gestures from the surgical team; and
a touchscreen interface configured to receive direct touch inputs,
the communication interface further providing an audible confirmation or visual indicator to the team upon successful receipt of a command.

5. The system of claim 1, wherein the contextual awareness module determines the surgical phase by analyzing sensor data indicative of team activities and tools in use, and wherein the cobotic system restricts or modifies its actions based on the identified phase such that, during critical surgical phases, non-essential cobot movements are minimized and task assistance is aligned with the surgical workflow.

6. The system of claim 1, wherein the shared information display subsystem includes at least one mounted display screen in the operating room and at least one augmented reality headset wearable by a team member, the shared information display subsystem being configured to present synchronized procedural information, instrument status updates, and team coordination cues in real time to all pertinent members of the surgical team.

7. The system of claim 1, wherein the safety and compliance control subsystem comprises:
a collision avoidance mechanism that automatically halts or re-routes cobot movement upon detecting a person or obstacle within a predefined safety radius;
a sterile-field recognition module that identifies a sterile field zone and prevents the cobot from entering or crossing into the sterile field; and
a proximity alert component that issues at least one of an audible alarm or a visual warning when the cobot comes within a threshold distance of a human team member or a sterile field boundary.

8. The system of claim 1, wherein the learning and adaptation engine employs machine learning algorithms to update task assignment rules and behavioral parameters based on data from multiple surgical procedures, thereby allowing the system to adapt to particular team behaviors or surgeon preferences over time, including recognizing preferred instrument handoff techniques and adjusting timing of assistance tasks according to past feedback.

9. The system of claim 1, wherein the audit trail and performance metrics module logs for each support task a timestamp, the identity of the task (including which instrument was passed or which adjustment was made), and the role of the human team member involved, and wherein the postoperative collaboration report generated by the module includes metrics of team efficiency including task completion times, periods of cobot inactivity, and instances where the cobot preemptively assisted an overburdened team member.

10. The system of claim 1, wherein the coordination module is configured to communicate with a surgical scheduling system and a hospital inventory system to verify, prior to the scheduled start time, that each required staff member has checked in and each required surgical tool or tray is available, and upon detecting that a required person or item is missing or delayed, the coordination module issues a notification alert to relevant personnel to facilitate prompt attendance or delivery.

11. A method for enhancing surgical team collaboration in a perioperative environment using a cobotic system, the method comprising:
positioning a cobot, including a mobile base and at least one articulated robotic arm with attached tools, within an operating room alongside a human surgical team;
monitoring activity of the surgical team in real time by capturing visual data, audio input, and spatial positions to detect the presence and location of team members and to identify each team member’s role;
dynamically allocating and performing support tasks with the cobot by analyzing predefined role responsibilities and real-time workload, and assigning the cobot to assist with tasks such as instrument delivery, lighting adjustment, or instrument tray management based on the needs of the team;
receiving multimodal commands from the surgical team by processing verbal instructions, recognizing hand gestures, or accepting inputs on a touchscreen interface, and providing corresponding audio-visual feedback from the cobot confirming that the commands have been understood and executed;
identifying a current surgical phase of the procedure by evaluating contextual cues from the operating room and adjusting the cobot’s behavior to align with the workflow requirements of the identified phase;
displaying shared information and cues by projecting or presenting surgical data, instrument status, and team coordination prompts on one or more local screens and augmented reality headsets, thereby keeping all team members informed of operative status and upcoming needs;
enforcing safety protocols by automatically navigating and operating the cobot in a manner that avoids collisions with personnel, respects sterile field boundaries, and issues proximity alerts to the team when necessary, in compliance with operating room safety standards;
adapting to team preferences by updating an internal learning model using data gathered from the ongoing procedure and previous procedures, such that the cobot refines its task allocation and timing to better suit the specific surgical team’s workflows and preferences;
logging collaborative interactions between the cobot and the human team throughout the procedure, including recording commands given, tasks performed, and timing information; and
generating a postoperative collaboration report that aggregates the logged data into performance metrics and event timelines, to facilitate review of team efficiency and identification of workflow improvements.

12. The method of claim 11, wherein monitoring the activity of the surgical team comprises using at least one depth-sensing camera to track positions of team members and using a pattern recognition algorithm to distinguish individuals, further associating each detected individual with a role by comparing their location and actions to expected role behaviors for a surgeon, anesthesiologist, or nurse.

13. The method of claim 11, wherein dynamically allocating and performing support tasks includes detecting when a particular team member is overburdened or has requested assistance and in response assigning the cobot to perform a support task for that team member, including passing a needed surgical instrument directly into the surgeon’s hand upon a verbal request, adjusting an overhead light in response to a gesture from the surgeon or staff, or pre-arranging instruments on a tray when the scrub nurse is occupied.

14. The method of claim 11, wherein receiving multimodal commands comprises interpreting spoken surgical commands via natural language processing of audio captured by the cobot’s microphones, and detecting hand gesture commands by analyzing visual data from the cobot’s cameras, and wherein providing audio-visual feedback includes audibly acknowledging a command with a confirmation tone or phrase and displaying a visual confirmation on a cobot-mounted indicator or connected display.

15. The method of claim 11, wherein enforcing safety protocols includes automatically halting movement of the cobot’s arm if a team member comes within a predefined safety zone around the cobot, and maintaining a virtual boundary corresponding to the sterile field such that the cobot refrains from extending any tool or component into a region designated as sterile, thereby preserving sterile conditions throughout the procedure.

16. The method of claim 11, wherein adapting to team preferences further comprises analyzing the timing and sequence of support tasks recorded over multiple procedures to personalize the cobot’s assistance, whereby the cobot anticipates routine needs of a specific surgical team reducing response time and improving team workflow in subsequent procedures.

17. A cobotic system configured to coordinate surgical team readiness for an operation, the system comprising:
a cobot including a mobile base and at least one articulated robotic arm equipped with sensors for operating within an operating room;
a coordination module in communication with a surgical scheduling system and configured to determine whether each designated surgical staff member and each required surgical instrument or supply is present in the operating room at a predetermined time relative to a scheduled start of a surgical procedure; and
a notification subsystem coupled to the coordination module and configured to issue an alert or instruction upon detecting that a required staff member or tool is not present by the predetermined lead time, thereby prompting corrective action to prevent idle delays before the surgical procedure begins.

18. The system of claim 17, wherein the coordination module interfaces with hospital information systems by accessing an electronic surgery schedule and querying personnel tracking data and inventory databases to verify attendance of specific staff and availability of requisite tools, flags any deficiencies in real time, and updates the operating room staff via electronic communication if an issue is detected.

19. The system of claim 17, wherein the notification subsystem is configured to send a reminder or escalation alert to designated personnel when a required surgical team member has not arrived in the perioperative area within a threshold time before the scheduled start, thereby ensuring humans are aware of and can address potential delays.

20. The system of claim 17, wherein the cobot is further configured, in response to the coordination module detecting a missing surgical tool or instrument prior to surgery start, to autonomously retrieve the missing instrument from a storage location or sterile supply area and deliver it to the operating room, thereby ensuring all required tools are on hand by the time the surgical procedure is set to commence.
